(19) United States
(12) Patent Application Publication
(10) Pub. No.: US 2007/0092523 A1
(43) Pub. Date: Apr. 26, 2007

(54) Combination Therapy Of Respiratory Diseases Using Antibodies

(75) Inventor: Leslie S. Johnson, Darnestown, MD (US)

Correspondence Address: JOHNATHAN KLEIN-EVANS, ONE MEDIMMUNE WAY, GAITHERSBURG, MD, 20878, US

(73) Assignee: MEDIMMUNE, INC., Gaithersburg, MD (US)

(21) Appl. No.: 11/562,676

(22) Filed: Nov. 22, 2006

Related U.S. Application Data
(63) Continuation of application No. 11/194,765, filed on Aug. 2, 2005.
Application No. 11/194,765 is a continuation of application No. 09/848,390, filed on May 3, 2001, now abandoned.
(60) Provisional application No. 60/201,402, filed on May 3, 2000.

(51) Int. Cl. A61K 39/42; A61K 31/7056; A61K 31/13

(52) U.S. Cl. 424/159.1; 514/43; 514/662

(57) ABSTRACT

Therapeutically effective anti-microbial compositions, useful especially against respiratory diseases caused or mediated by viruses, bacteria, and other respiratory parasites are disclosed, wherein said compositions comprise at least one neutralizing antibody, including high affinity antibodies, and an additional anti-infectious agent, such as an antiviral agent, for example, ribavirin, amantadine, rimantadine, or a neuraminidase-inhibitor. or antibacterial agents, including other antibodies. Also disclosed are methods of using such compositions to treat and/or prevent respiratory and related diseases.

This application claims priority based on U.S. Provisional Application 60/201,402, filed 3 May 2000, the disclosure of which is hereby incorporated by reference in its entirety.

### FIELD OF THE INVENTION

The present invention relates to novel compositions of antibodies and anti-microbial agents useful in the treatment and/or prevention of respiratory and related diseases.

### BACKGROUND OF THE INVENTION

The current incidence of infection caused by resistant or difficult to control microbes, including both viruses and bacteria, has created a need for newer approaches to controlling such organisms, as well as to treating those already infected.

Among the more difficult infectious agents to control and treat are the viruses. For example, respiratory syncytial virus (RSV) is a major cause of acute respiratory illness in young children admitted to hospitals and the major cause of lower respiratory tract infection in young children. A major obstacle to producing an effective vaccine against such agents as RSV has been the issue of safety. Conversely, the use of immunoglobulins against such viral agents has proven of some value. For example, studies have shown that high-titred RSV immunoglobulin was effective both in prophylaxis and therapy for RSV infections in animal models.

Bacteria also present a formidable challenge in the area of disease control and prevention. This is especially true with the rise of nosocomial infections in hospitals and elsewhere. Thus, the use of high-titred antibodies in controlling such infections would be a welcomed solution to this problem.

As a result, an alternative approach to microbial therapy has been the development of antibodies, especially neutralizing monoclonal antibodies, with high specific neutralizing activity. One drawback to this route has been the need to produce human antibodies rather than those of mouse or rat and thus minimize the development of human anti-mouse or anti-rat antibody responses, which potentially results in further immunopathology.

One alternative approach has been the production of antibodies in which the genes encoding the mouse heavy and light chain variable regions have been coupled to the genes for human heavy and light chain constant regions to produce chimeric, or hybrid, antibodies.

In some cases, mouse CDRs have been grafted onto human constant and framework regions with some of the mouse framework amino acids being substituted for correspondingly positioned human amino acids to provide a “humanized” antibody. [U.S. Pat. Nos. 5,693,761 and 5,693,762]

A humanized anti-RSV antibody with good affinity has been prepared and is currently being marketed.

In addition, a number of other therapeutic agents useful against such viruses as respiratory syncytial virus (RSV), as well as parainfluenza virus (PIV), have made their appearance. However, some of these chemical agents, such as ribavirin, have presented drawbacks. Thus, for example, ribavirin, although currently licensed for therapy of RSV pneumonia and bronchiolitis (Hall et al, N. Engl. J. Med., 308: 1443 (1983); Hall et al., JAMA, 254:3047 (1985), is still of controversial value and has to be administered over an 18 hour period by aerosol inhalation. In addition, the level of secondary infection following cessation of treatment is significantly higher than in untreated patients.

### BRIEF SUMMARY OF THE INVENTION

The present invention is directed to compositions comprising a monoclonal antibody, especially a neutralizing antibody against respiratory viruses, especially respiratory syncytial virus, as well as other therapeutic agents useful in the treatment of respiratory disease.

In accordance with an aspect of the present invention, there are provided therapeutic compositions containing a neutralizing antibody as well as one or more additional antiviral agents capable of working either separately or in concert to treat and/or prevent antiviral infections, especially those of the respiratory system, most especially diseases caused by RSV.

In one embodiment, the therapeutic composition of the present invention comprises an anti-RSV antibody useful in treating and/or preventing virally induced respiratory disease, and an additional antiviral agent useful against RSV.

In a separate embodiment, the present invention is also directed to compositions comprising an anti-RSV antibody, including high affinity antibodies (wherein the term high affinity means an antibody having an affinity, or dissociation constant with antigen, of about 10⁻⁹ M or lower), and an additional anti-infectious agent, the latter being effective against infections accompanying that caused by RSV, such as infections by other viruses, for example, parainfluenza virus, influenza A, influenza B and influenza C, as well as by bacteria, fungi, and various other parasites.

In a preferred embodiment, a neutralizing monoclonal antibody used in the compositions of the present invention is an antibody whose variable sequences are disclosed in FIGS. 7 and 8 of U.S. Pat. No. 5,824,307 or Medi-493 in Johnson et al, J. of Infectious Diseases, 176, 1215-1224 (1997) (the disclosures both of which are hereby incorporated by reference in its entirety). The use of structural variants of this antibody are also specifically contemplated by the present invention.

In one preferred embodiment, a therapeutic composition of the present invention comprises an anti-RSV neutralizing antibody, including high affinity antibodies, most preferably an antibody specific for the F epitope of RSV, or a variant thereof, including active fragments thereof, and an antiviral agent having therapeutic value in the treatment of viral diseases of the respiratory system, preferably diseases caused by RSV, or even PIV.

In specific embodiments of the present invention, the antiviral agent is ribavirin amantadine, rimantadine, or a neuraminidase-inhibitor.

In another most preferred embodiment of the present invention, the therapeutic composition comprises an anti-RSV antibody, including high affinity antibodies, an anti-Interleukin-6 (anti-IL-6) antibody and a non-antibody antiviral agent, such as ribavirin, amantadine, rimantadine, or a neuraminidase-inhibitor.

In another embodiment of the present invention there is provided a composition for treating and/or preventing bacterial induced diseases, especially bacterial diseases affecting the respiratory system.

### DETAILED SUMMARY OF THE INVENTION

One problem facing clinicians in their attempts to treat microbial, including both virus and bacteria, caused infections has been the extremely toxic nature of many antimicrobial agents, especially those used to combat viral infections, such as respiratory infections, especially agents like ribavirin.

The compositions and treatments afforded according to the present invention represents a solution to this problem by offering compositions and treatments that take advantage of the unique abilities of antibodies, especially neutralizing antibodies, most especially high affinity, high specificity neutralizing antibodies such as those utilized herein, to control the ravages of bacterial and viral infections, most especially as they affect the delicate tissues of the respiratory system, and thereby offset the otherwise deleterious effects of relying solely on highly potent, and potentially toxic, antimicrobial agents that must, because of their chemical and biological properties, perforce be administered in sparing, and sometimes less than effective, dosages.

More specifically, the availability of compositions containing reduced amounts of such potent drugs along with accompanying antibodies, including high affinity antibodies, would serve to provide a middle ground for treatment and/or prevention of viral-induced diseases, such as those of the respiratory system, especially those caused by RSV. In addition, such compositions could contain one or more other chemical agents also effective, to varying degrees, against the viral agents in question. This would be desirable from the point of view of the neutralizing ability of such antibodies coupled with the presence of other chemical therapeutic agents as a means of reducing any potentially undesirable side effects of both types of agent while at the same time providing increased effectiveness due to a multi-stage attack on the organisms in question using agents whose mechanism of action is sufficiently diverse to avoid unwanted cross-reactions and other interfering effects.

The present invention is directed to therapeutically effective compositions comprising a neutralizing monoclonal antibody, including high affinity neutralizing antibodies, against respiratory viruses, such as respiratory syncytial virus (RSV), and even parainfluenza virus (PIV), influenza A, B, and C, as well as related viral agents causing respiratory disease, and other therapeutic agents, including other antibodies and non-antibody agents, useful in the treatment of respiratory disease.

It is thus an object of the present invention to provide therapeutic compositions comprising one or more neutralizing antibodies, including high affinity neutralizing antibodies, especially an anti-RSV antibody, most especially a high affinity antibody with the same antigenic specificity of an antibody such as Medi-493, and active variants and fragments thereof, as well as one or more additional agents capable of working either separately or in concert to treat and/or prevent antiviral infections, or otherwise combat and/or relieve the deleterious physiological and/or immunological effects of such infections, especially infections of the respiratory system, most especially diseases caused by RSV, or even PIV, or other viruses, as well as bacterial agents.

Thus, the present invention relates to a composition comprising a therapeutically effective amount of an antibody, including active variants and fragments thereof, having specificity for one or more epitopes of respiratory syncytial virus (RSV), and at least one additional antiviral agent wherein said antibody and agent are suspended in a pharmacologically acceptable carrier, diluent or excipient.

The anti-viral antibody, such as a high affinity antibody with the same antigenic specificity of an antibody as disclosed in U.S. Pat. No. 5,824,307, especially the antibody whose heavy and light chain variable sequences are disclosed in FIGS. 7and 8, respectively, thereof, or Medi-493, and active variants and fragments thereof, useful in the present invention can include a whole antibody molecule (i.e., a tetrameric structure with the common H₂L₂ arrangement) or active fragments thereof. Such fragments include, but are not limited to, Fab, F(ab′)₂, single chain antibodies, chimeric antibodies, such as human-chimeric antibodies, humanized antibodies, the latter being formed from human framework and constant regions with complementarity determining regions (CDRs) derived from a species other than human, such as murine, as well as completely synthetic (i.e., recombinant) antibodies having amino acid sequences different from those of any antibody produced in nature or thus far created by man. Such wholly synthetic antibodies may be produced by cloning in recombinant cells produced for such purposes or by direct chemical synthesis in vitro. These can also include wholly human antibodies formed by combination of framework and CDR sequences derived from different human antibodies.

The anti-viral, e.g. anti-RSV, antibody of the present invention can also include antibody molecules, and active fragments thereof, having a different amino acid sequence from an antibody disclosed such as the aforementioned Medi-493 (in U.S. Pat. No. 5,824,307 and thus be a variant thereof) so long as high affinity for the respiratory virus, such as RSV, is maintained, or other microbe, including bacteria, is maintained.

In accordance with the present invention, the neutralizing antibodies useful in the methods disclosed herein typically have affinity constants for their respective antigenic epitopes that are in the range of no greater than about 1 nM (or at least about 10⁻⁹ M). Because such high affinities are not easily measured, except by the procedures described herein, such value may commonly be considered as part of a range and may, for example, be within 2 fold of the nM values recited herein. Thus, they may be about 2 fold greater or lower than this value of may equal this value and still be useful in the present invention. Because this is a dissociation constant, the higher the value, the greater the degree of dissociation of the antigen and antibody and thus the lower the affinity. Such values may be easily converted to association constants by taking the reciprocal of the dissociation constant and adjusting the units to reciprocal molar in place of molar. In such case, the affinity of the antibody for its antigen will increase with increasing association constants. Such neutralizing antibodies are known in the art (see, for example, antibodies disclosed in FIGS. 7 and 8 of U.S. Pat. No. 5,824,307 where the affinity is denoted by a dissociation constant, which is in the nature of a binding constant, so as to give units of molarity). As such, the affinity of the antibody for antigen is inversely proportional to the value of this constant (i.e., the higher the constant, the lower the affinity). Such a constant is readily calculated from the rate constants for the association-dissociation reactions as measured by standard kinetic methodology for antibody reactions (see U.S. Pat. No. 5,824,307 or Johnson et al, J. of Infectious Diseases, 176, 1215-1224 (1997) for a suggested method of doing this).

The compositions of the present invention are not limited in their mode of administration to the patient. Thus, such administration can include parenteral as well as oral administration, and thus include intravenous, intramuscular, pulmonary and nasal administration. In addition, for purposes of administration, such compositions can be in the form of an aerosol or other type of spray, especially a fine particle aerosol, as defined below. However, because of the nature of the diseases to be controlled and the types of chemical entities making up the present compositions, a preferred mode of administration is directly through the respiratory system. The antiviral agents contemplated for use in the compositions of the present invention are commonly administered through the respiratory system, often in the form of an aerosol.

In other embodiments, the composition of the present invention comprises an anti-RSV antibody, including high affinity antibodies, in addition to an antibody whose specificity is directed toward some other viral agent. Such embodiments include compositions comprising additional high-affinity anti-RSV antibodies. A specific embodiment of such a composition comprises an antibody such as Medi-493 (as disclosed in Medi-493 in Johnson et al, J. of Infectious Diseases, 176, 1215-1224 (1997) and an additional anti-RSV antibody, including a high affinity antibody.

The compositions of the present invention also comprise a non-antibody antiviral agent. Such compositions may include a single antiviral agent or two or more antiviral agents, either at similar or different concentrations and dosages, depending on the effectiveness of the agent against the virus in question as well as on the needs of the patient and the determinations and inclination of the clinician, in whose sound discretion such decisions are left. In some embodiments, the antiviral agent is ribavirin, amantadine, rimantadine, or a neuraminidase-inhibitor, or an analog of one of these or a therapeutically effective agent whose chemical structure incorporates all or part of the anti-viral molecule. For purposes of the present invention, the term “therapeutically effective” means any agent having antiviral activity, especially an agent approved for commercial use as an antiviral agent and for use in treating and/or preventing viral diseases in animals, especially in humans.

In a preferred embodiment of the present invention, the antimicrobial agent is the antiviral agent ribavirin. Ribavirin is a purine nucleoside analog exhibiting inhibition of a wide range of RNA and DNA viruses, including respiratory syncytial virus, the latter being inhibited at in vitro concentrations of 3 to 10 μg/ml. In general it can be given orally whereupon its bioavailability is about 45% with peak concentrations in plasma after about 1 to 2 hours. Single adult doses are in the 600 to 1200 mg range. The general route of administration for ribavirin is by aerosol with a dose to infants of about 1.4 mg/kg of body weight per hour and treatment for about 12 to 18 hours per day over a 3 to 7 day period. As a result, use of such antiviral agents in conjunction with antibodies as set forth in the present disclosure provide an advantageous means of decreasing the dosages required for the antiviral agents, such as ribavirin, while still maintaining high levels of therapeutic efficiency.

The pathology due to viral agents such as RSV is due to both direct tissue destruction and inflammation due to recruitment of immune cells. Agents like ribavirin have limited antiviral properties but may serve to limit RSV pathology by altering TH1/TH2 responses. Thus, combination of agents such as ribavirin with an authentic anti-RSV agent, such as an anti-RSV antibody, for example an antibody having specificity similar, if not identical, to an antibody such as Medi-493 (as disclosed in Medi-493 in Johnson et al, J. of Infectious Diseases, 176, 1215-1224 (1997) or U.S. Pat. No. 5,824,307), or active fragments thereof, are thus highly effective in the treatment of RSV.

Pharmaceutical compositions will comprise sufficient active antibody and antiviral agents, so as to produce a therapeutically effective amount of the composition, i.e., an amount sufficient to reduce the amount of infecting virus, for example, RSV. The pharmaceutical compositions will also contain a pharmaceutically acceptable carrier, which includes all kinds of diluents and/or excipients, which include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. A thorough discussion of pharmaceutically acceptable excipients is available in REMINGTON'S PHARMACEUTICAL SCIENCES (Mack Pub. Co., N.J. 1991).

The present invention is also directed to methods of treating and/or preventing a respiratory disease, especially diseases caused by respiratory syncytial virus, including diseases like bronchiolitis, comprising administering to an animal, especially a human patient, at risk thereof, or afflicted therewith, of a therapeutically effective amount of a composition selected from the group consisting of the compositions disclosed herein.

Thus, the present invention provides a method for treating an animal, especially a human patient, suffering from a lower respiratory disease, such as RSV, and wherein said disease is caused by a viral agent or bacterial agent, including cases where said microbial agent is not the main cause of distress but merely serves to exacerbate an already existing condition, such as by causing clinical complications thereof, including instances of superinfection. The compositions of the present invention may be administered in the form of an aerosol spray of fine particles. The compositions of the present invention may be administered directly to the lower respiratory tract (for treating children) or to the upper respiratory tract (for treating adults) by intra-nasal spray. Such sprays must be formed of fine particles, which includes pharmacologically acceptable particles containing a therapeutically active amount of the compositions disclosed herein, and wherein such particles are no larger than about 10 μm in diameter, preferably no larger than about 5 μm in diameter and most preferably no larger than about 2 μm in diameter.

Optimum dosages for the anti-RSV antibodies making up the compositions of the present invention may be in the range of 5 to 20 mg/kg of body weight, the optimum for antibodies such as Medi-493 [as disclosed in U.S. Pat. No. 5,824,307 or in Johnson et al, J. of Infectious Diseases, 176, 1215-1224 (1997)] being about 15 mg/kg of body weight (when given intravenously). The non-antibody antiviral agents used in said compositions, other than the antiviral antibodies employed herein are commonly in the range of about 1 μg to about 1 gram per kg body weight.

An example of a primary infectious agent to be controlled by the compositions and methods of the present invention is respiratory syncytial virus but it is possible that other infectious agents may also be present as opportunistic pathogens. These can include other viruses, especially influenza A, influenza B, and influenza C, and parainfluenza virus (PIV), especially PIV3, some variant or mutant of RSV, a respiratory corona virus and even and adenovirus, and various types of bacterial agents that are either sources or primary infection within the respiratory system or else are agents capable of aggravating existing viral diseases or else weakening the respiratory system so as to make it more susceptible to such viral diseases.

The additional infectious agents acting as opportunistic pathogens are not limited to the viruses and bacteria. Thus, additional infection may be caused by non-viral or bacterial organisms, including various fungi and other parasites. As a result, the compositions according to the present invention may also comprise anti-infectious agents other than antiviral agents. Therapeutically active compositions within the present invention may thus comprise an anti-RSV antibody and an antibacterial agent, including antibiotics, as well as antifungal agents and antiparasitic agents of a broad or narrow spectrum. In addition, all of the latter additional agents may themselves be low or high affinity polyclonal or monoclonal antibodies with specificity against bacteria, or fungi, or other parasites infecting the respiratory system, as well as other related or unrelated systems.

The compositions disclosed according to the present invention for therapy of diseases as recited herein can easily include multiple antibodies against the same or different viruses, or against a virus and an addition microbial infectious agent, or against some non-viral microbial infectious agent, and may additionally include non-immunological agents in combination with said antibodies. In specific embodiments of the present invention, compositions disclosed herein may include an antibody against a virus, such as RSV, plus an antibody against a bacterial agent, especially one that infects the respiratory system, such as that causing tuberculosis, and, optionally, an antiviral agent. A therapeutic composition within the present invention may likewise comprise an anti-viral antibody, a non-immunological anti-viral agent, such as ribavirin, amantadine, rimantadine, or a neuraminidase-inhibitor, where RSV is the primary infectious agent, and an antimicrobial agent effective in the treatment of some non-viral pathogen, such as bacteria, including the agent for tuberculosis, or against some parasitic agent.

Thus, in accordance with a highly specific embodiment of the present invention, the anti-infectious agent used in composition with an anti-RSV antibody, including high affinity antibodies, may be an anti-bacterial agent, including but not limited to a macrolide, a penicillin, a cephalosporin, or a tetracycline, or may be an antifungal agent, including but not limited to amphotericin b, fluconazole, or ketoconazole, or an anti-parasitic agent, including but not limited to trimethoprim, pentamidine, or a sulfonamide. The anti-infectious agent may be an anti-viral agent such as ribavirin, amantadine, rimantadine, or a neuraminidase-inhibitor. Such additional agents can also include agents useful against other viruses as well as other agents useful against RSV.

However, in all highly preferred embodiments of the present invention the primary disease to be treated and/or prevented using the compositions disclosed herein is caused by respiratory syncytial virus (RSV).

With the advent of methods of molecular biology and recombinant technology, it is now possible to produce antibodies for use in the present invention by recombinant means and thereby generate gene sequences that code for specific amino acid sequences found in the polypeptide structure of the antibodies. This has permitted the ready production of antibodies having sequences characteristic of neutralizing antibodies from different species and sources.

In accordance with the foregoing, the antibodies useful in the methods of the present invention are anti-RSV antibodies, most preferably a antibodies whose specificity is toward the same epitope of RSV as Medi-493 (U.S. Pat. No. 5,824,307) and include all therapeutically active variants and fragments thereof whether produced by recombinant methods or by direct synthesis of the antibody polypeptides.

The anti-RSV antibodies, including high affinity antibodies, useful in the compositions of the present invention will commonly comprise a mammalian, preferably a human, constant region and a variable region, said variable region comprising heavy and light chain framework regions and heavy and light chain CDRs, wherein the heavy and light chain framework regions are derived from a mammalian antibody, preferably a human antibody, and wherein the CDRs are derived from an antibody of some species other than a human, preferably a mouse. Where the framework amino acids are also derived from a non-human, the latter is preferably a mouse.

In addition, antibodies of the invention, including high affinity antibodies, bind the same epitope as the antibody from which the CDRs are derived, and wherein at least one of the CDRs of said antibody, including high affinity antibodies, contains amino acid substitutions, and wherein said substitutions comprise the replacement of one or more amino acids in the CDR regions by non-identical amino acids, preferably the amino acids of the correspondingly aligned positions of the CDR regions of the human antibody contributing the framework and constant domains.

The contemplated host intended for treatment or prophylaxis with the compositions disclosed herein is generally an animal, especially a mammal, most especially a human patient.

Another preferred embodiment of the invention provides a method of treating upper and/or lower respiratory tract diseases in a host, especially that caused by respiratory syncytial virus, susceptible to or suffering from such disease, comprising administering to the host a therapeutically effective amount of a composition comprising an antibody, preferably an anti-RSV antibody, most preferably the antibody whose variable heavy and light chain sequences are disclosed in FIGS. 7 and 8 of U.S. Pat. No. 5,824,307, including therapeutically active variants and fragments thereof, an anti-viral agent other than the previously stated antibody, with activity against RSV and an anti-inflammatory agent, said composition being sufficiently active as to produce a therapeutic effect against said disease or to protect against said disease. Such diseases include all manner of respiratory diseases, especially those caused by, or complicated by, RSV infections. Thus, the antimicrobial compositions of the present invention are also useful against other microbial agents besides RSV, especially where such other microbial agents, such as viruses or bacteria and the like, act as opportunistic agents to aggravate an already existing infection, such as an RSV infection, or where the presence of such non-RSV agent acts to make treatment of the respiratory infection more difficult. Of course, the clinical use of any composition of the present invention is a clinical decision to be made by the clinician and the exact course of such treatment is left to the clinician's sound discretion, with all such courses of treatment deemed within the bounds of the present invention.

Said composition may be administered by any available means, including but not limited to, oral, intravenous, intramuscular, pulmonary and nasal routes, and wherein said composition is present as a solution, a suspension or an aerosol spray, especially of fine particles. Such composition may be administered directly to the upper or lower respiratory tract of the host. The virus to be treated is respiratory syncytial virus, but other viruses may be treated simultaneously, such as parainfluenza virus, especially type 3, influenza A, influenza B and influenza C. In accordance with the methods of treatment disclosed herein, the non-antibody anti-viral agent may be ribavirin, amantadine, rimantadine, or a neuraminidase-inhibitor. Such compositions can also include an immunoglobulin, such as human immunoglobulin G, which comprises antibodies against RSV or some other opportunistic virus.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned are incorporated herein by reference. Unless mentioned otherwise, the techniques employed or contemplated herein are standard methodologies well known to one of ordinary skill in the art. All materials, methods, and examples are illustrative only and not limiting.

What is claimed is:

1. An anti-microbial composition comprising a therapeutically effective amount of at least one anti-microbial neutralizing antibody, including therapeutically active variants and fragments thereof, and at least one additional anti-microbial agent wherein said neutralizing antibody and said additional anti-microbial agent are suspended in a pharmacologically acceptable carrier.

2. The anti-microbial composition of claim 1 wherein said neutralizing antibody is an anti-viral antibody.

3. The anti-microbial composition of claim 1 wherein said anti-microbial agent is an anti-viral agent.

4. The anti-microbial composition of claim 1 wherein said anti-microbial neutralizing antibody is an anti-viral neutralizing antibody and said anti-microbial agent is an antiviral agent.

5. The anti-microbial composition of claim 1 wherein said anti-microbial agent is a member selected from the group consisting of ribavirin, amantadine, rimantadine, and neuraminidase-inhibitors.

6. The anti-microbial composition of claim 2 wherein said neutralizing antibody has affinity for the F epitope of respiratory syncytial virus.

7. The anti-microbial composition of claim 1 wherein said composition comprises at least 2 anti-microbial neutralizing antibodies of differing specificity, including therapeutically active variants and fragments thereof.

8. The anti-microbial composition of claim 7 wherein said anti-microbial neutralizing antibodies comprise at least one anti-viral antibody and at least one antibody with specificity for an epitope found on a non-viral microbe.

9. The anti-microbial composition of claim 7 wherein said anti-microbial neutralizing antibodies are selected from the group consisting of antiviral, antibacterial, anti-fungal, and anti-parasitic antibodies.

10. The anti-microbial composition of claim 9 wherein said anti-microbial antibodies comprise at least one antiviral antibody.

11. The anti-microbial composition of claim 7 wherein said composition comprises at least 2 antiviral antibodies.

12. The anti-microbial composition of claim 11 wherein at least one of said antibodies is an antibody with respiratory syncytial virus neutralizing activity.

13. The anti-microbial composition of claim 1 wherein said additional anti-microbial agent is antibody.

14. The anti-microbial composition of claim 13 wherein said additional anti-microbial antibody has specificity for a microbe other than a virus.

15. The anti-microbial composition of claim 1 wherein said anti-microbial neutralizing agent is antibody.

16. The composition of claim 2 wherein the anti-microbial agent is amantadine.

17. The composition of claim 1 wherein said additional anti-microbial agent is an anti-bacterial agent.

18. The composition of claim 1 wherein said additional anti-microbial agent is an anti-fungal agent.

19. The composition of claim 1 wherein said additional anti-microbial agent is an anti-parasitic agent.

20. The composition of claim 1 wherein said neutralizing antibody is a high affinity neutralizing antibody.

21. A method of treating a respiratory disease caused by a microbial agent comprising administering to a patient afflicted therewith a therapeutically effective amount of the composition of claim 1.

22. The method of claim 21 wherein said microbial agent is a virus.

23. The method of claim 23 wherein said virus is respiratory syncytial virus (RSV).

24. A method of protecting against a respiratory disease caused by a microbial agent comprising administering to a patient at risk thereof a therapeutically effective amount of the composition of claim 1.

25. The method of claim 24 wherein said microbial agent is a virus.

26. The method of claim 25 wherein said virus is respiratory syncytial virus (RSV).

27. The composition of claim 1 wherein said antibody is MEDI-493.

28. The process of claim 21 wherein the antibody in said composition of claim 1 is MEDI-493.

29. The process of claim 24 wherein the antibody in said composition of claim 1 is MEDI-493.
